(19) United States
(12) Patent Application Publication
(10) Pub. No.: US 2011/0071439 A1
(43) Pub. Date: Mar. 24, 2011

(54) Tactile input system

(75) Inventors: Paul Bach-Y-Rita, Madison, WI (US); Edward N. Fisher, Middleton, WI (US); Juana Esther Bach-Y-Rita, Madison, WI (US)

(73) Assignee: WICAB, INC., Middleton, WI (US)

(21) Appl. No.: 10/577,282

(22) PCT Filed: Oct. 22, 2003
(86) PCT No.: PCT/US2004/035305

§ 371 Date: Nov. 20, 2007

(30) Foreign Application Priority Data
Oct. 22, 2003 (US) 60531915

(51) Int. Cl. A61H 1/00; A61N 1/36; A61F 7/00

(52) U.S. Cl. 601/1; 607/2; 607/96

(57) ABSTRACT

The present invention relates to systems and methods for management of brain and body functions and sensory perception. For example, the present invention provides systems and methods of sensory substitution and sensory enhancement (augmentation) using tactile stimulators implanted under the skin.

The present invention claims priority to U.S. Provisional Application Ser. No. 60/531,915, filed Oct. 22, 2003.

### FIELD OF THE INVENTION

The present invention relates to systems and methods for management of brain and body functions and sensory perception. For example, the present invention provides systems and methods of sensory substitution and sensory enhancement (augmentation) using tactile stimulators implanted under the skin.

### BACKGROUND OF THE INVENTION

The mammalian brain, and the human brain in particular, is capable of processing tremendous amounts of information in complex manners. The brain continuously receives and translates sensory information from multiple sensory sources including, for example, visual, auditory, olfactory, and tactile sources. Through processing, movement, and awareness training, subjects have been able to recover and enhance sensory perception, discrimination, and memory, demonstrating a range of untapped capabilities. What are needed are systems and methods for better expanding, accessing, and controlling these capabilities.

### DESCRIPTION OF DRAWINGS

FIG. 1 is a simplified perspective view of an exemplary input system wherein an array of transmitters 104 magnetically actuates motion of a corresponding array of stimulators 100 implanted below the skin 102.

FIG. 2 is a simplified cross-sectional side view of a stimulator 200 of a second exemplary input system, wherein the stimulator 200 delivers motion output to a user via a deformable diaphragm 212.

FIG. 3 is a simplified circuit diagram showing exemplary components suitable for use in the stimulator 200 of FIG. 2.

### SUMMARY OF THE INVENTION

The present invention provides tactile input systems that reduce or eliminate many of the problems encountered in prior systems by providing stimulators that are implanted beneath the epidermis or otherwise positioned under the skin or other tissues. One advantage of such a system is the ability to substantially reduce size of the stimulators because their output is closer to the nerves of the skin (or other tissue) and is no longer “muffled.” Such size reduction allows higher stimulator densities to be achieved. Additionally, interconnectivity problems, and issues inherent in providing input signals from an external camera, microphone, or other input device to an internal/subdermal stimulator (i.e., the need to provide leads extending below the skin), may be avoided by providing one or more transmitters outside the body, and preferably adjacent the area of the skin where the stimulator(s) are embedded, which wirelessly provide the input signals to the embedded stimulator(s).

Thus, in some embodiments, the present invention provides a tactile input system comprising one or more stimulators implanted in the skin of a subject (e.g., below the epidermis in a closely spaced array), wherein the stimulators or a portion thereof are independently configured to deliver a tactile stimulation (e.g., mechanical, electrical, thermal stimulation). In some embodiments, the stimulators are configured to provide the tactile stimulation in response to a wireless signal (e.g., communicated via a light signal). In preferred embodiments, the stimulators are provided with a biocompatible coating or are constructed of biocompatible material. In some embodiments, the system further comprises a transmitter configured to transmit a signal to one or more of said stimulators to initiate said tactile stimulation. In some preferred embodiments, the stimulators individually have a volume of less than 10 cubic millimeters. In some embodiments, the stimulators comprise a movable diaphragm. In some embodiments, the stimulators are not in direct or indirect physical contact with each other. The system may further comprise external sensors and equipment (e.g., video sensors, audio sensors, environmental sensors, tactile sensors, heat sensors, chemical sensors, etc.), processors, or other useful components. The present invention further provides an implantable tactile input system comprising one or more stimulators configured to be implanted in the skin of a subject (e.g., below the epidermis in a closely spaced array), wherein the stimulators or a portion thereof are independently configured to deliver a tactile stimulation to the subject when implanted.

The present invention also provides methods for imparting information to a subject comprising the step of transmitting a signal from a transmitter to one or more stimulators implanted in the skin of said subject under conditions such that the stimulators provide a tactile stimulation that conveys information from the signal to the brain of the subject. In some embodiments, the information comprises visual, audio, tactile, or environmental information. In some embodiments, the information comprises tactile information from a body location other than the location where the stimulators are implanted.

### DEFINITIONS

To facilitate an understanding of the present invention, a number of terms and phrases are defined below:

As used herein, the term “subject” refers to a human or other vertebrate animal. It is intended that the term encompass patients.

As used herein, the term “amplifier” refers to a device that produces an electrical output that is a function of the corresponding electrical input parameter, and increases the magnitude of the input by means of energy drawn from an external source (i.e., it introduces gain). “Amplification” refers to the reproduction of an electrical signal by an electronic device, usually at an increased intensity. “Amplification means” refers to the use of an amplifier to amplify a signal. It is intended that the amplification means also includes means to process and/or filter the signal.

As used herein, the term “receiver” refers to the part of a system that converts transmitted waves into a desired form of output. The range of frequencies over which a receiver operates with a selected performance (i.e., a known level of sensitivity) is the “bandwidth” of the receiver.

As used herein, the term “transducer” refers to any device that converts a non-electrical parameter (e.g., sound, pressure or light), into electrical signals or vice versa.

As used herein, the terms “stimulator” and “actuator” are used herein to refer to components of a device that impart a stimulus (e.g., vibrotactile, electrotactile, thermal, etc.) to tissue of a subject. When referenced herein, the term stimulator provides an example of a transducer. Unless described to the contrary, embodiments described herein that utilize stimulators or actuators may also employ other forms of transducers.

The term “circuit” as used herein, refers to the complete path of an electric current.

As used herein, the term “resistor” refers to an electronic device that possesses resistance and is selected for this use. It is intended that the term encompass all types of resistors, including but not limited to, fixed-value or adjustable, carbon, wire-wound, and film resistors. The term “resistance” (R; ohm) refers to the tendency of a material to resist the passage of an electric current, and to convert electrical energy into heat energy.

As used herein, the term “electrode” refers to a conductor used to establish electrical contact with a nonmetallic part of a circuit, in particular, part of a biological system.

The term “housing” refers to the structure encasing or enclosing at least one component of the devices of the present invention. In preferred embodiments, the “housing” is produced from a “biocompatible” material. In some embodiments, the housing comprises at least one hermetic feedthrough through which leads extend from the component inside the housing to a position outside the housing.

As used herein, the term “biocompatible” refers to any substance or compound that has minimal (i.e., no significant difference is seen compared to a control) to no irritant or immunological effect on the surrounding tissue. It is also intended that the term be applied in reference to the substances or compounds utilized in order to minimize or to avoid an immunologic reaction to the housing or other aspects of the invention. Particularly preferred biocompatible materials include, but are not limited to titanium, gold, platinum, sapphire, stainless steel, plastic, and ceramics.

As used herein, the term “implantable” refers to any device that may be implanted in a patient. It is intended that the term encompass various types of implants. In preferred embodiments, the device may be implanted under the skin (i.e., subcutaneous), or placed at any other location suited for the use of the device (e.g., within temporal bone, middle ear or inner ear). An implanted device is one that has been implanted within a subject, while a device that is “external” to the subject is not implanted within the subject (i.e., the device is located externally to the subject's skin).

As used herein, the term “hermetically sealed” refers to a device or object that is sealed in a manner that liquids or gases located outside the device are prevented from entering the interior of the device, to at least some degree. “Completely hermetically sealed” refers to a device or object that is sealed in a manner such that no detectable liquid or gas located outside the device enters the interior of the device. It is intended that the sealing be accomplished by a variety of means, including but not limited to mechanical, glue or sealants, etc. In particularly preferred embodiments, the hermetically sealed device is made so that it is completely leak-proof (i.e., no liquid or gas is allowed to enter the interior of the device at all).

As used herein the term “processor” refers to a device that is able to read a program from a computer memory (e.g., ROM or other computer memory) and perform a set of steps according to the program. Processor may include non-algorithmic signal processing components (e.g., for analog signal processing).

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape, flash memory, and servers for streaming media over networks.

As used herein the terms “multimedia information” and “media information” are used interchangeably to refer to information (e.g., digitized and analog information) encoding or representing audio, video, and/or text. Multimedia information may further carry information not corresponding to audio or video. Multimedia information may be transmitted from one location or device to a second location or device by methods including, but not limited to, electrical, optical, and satellite transmission, and the like.

As used herein the term “in electronic communication” refers to electrical devices (e.g., computers, processors, communications equipment) that are configured to communicate with one another through direct or indirect signaling. For example, a conference bridge that is connected to a processor through a cable or wire, such that information can pass between the conference bridge and the processor, are in electronic communication with one another. Likewise, a computer configured to transmit (e.g., through cables, wires, infrared signals, telephone lines, etc) information to another computer or device, is in electronic communication with the other computer or device.

As used herein the term “transmitting” refers to the movement of information (e.g., data) from one location to another (e.g., from one device to another) using any suitable means.

### DETAILED DESCRIPTION OF THE INVENTION

A description of several exemplary versions of the implanted system follows. In preferred embodiments, the implantable stimulator(s) are implanted in the dermis, the skin layer below the epidermis (the outer layer of skin which is constantly replaced) and above the subcutaneous layer (the layer of cells, primarily fat cells, above the muscles and bones, also sometimes referred to as the hypodermis). Most tactile nerve cells are situated in the dermis, though some are also located in the subcutaneous layer. Therefore, by situating a stimulator in the dermis, the stimulator is not subject to the insulating effect of the epidermis, and more direct input to the tactile nerve cells is possible. Perceptible tactile mechanical (motion) inputs may result from stimulator motion on the order of as little as 1 micrometer, whereas above-the-skin tactile input systems require significantly greater inputs to be perceivable (with sensitivity also depending where on the body the system is located). If the stimulators use electrical stimulation in addition to or instead of mechanical (e.g., motion) stimulation, a problem encountered with prior electrotactile systems—that of maintaining adequate conductivity—is also reduced, since the tissue path between the stimulators and the tactile nerve cells is short and generally conductive. Additionally, so long as a stimulator is appropriately encased in a biocompatible material, expulsion of the stimulator from the skin is unlikely. In this respect, it is noted that when tattoos are applied to skin, ink particles (sized on the micrometer scale) are driven about ⅛ inch into the skin (more specifically the dermis), where they remain for many years (and are visible through the translucent, and oven nearly transparent, epidermis). In contrast, implantation in the epidermis would cause eventual expulsion, since the epidermis is constantly replaced. However, expulsion may be desired for certain application.

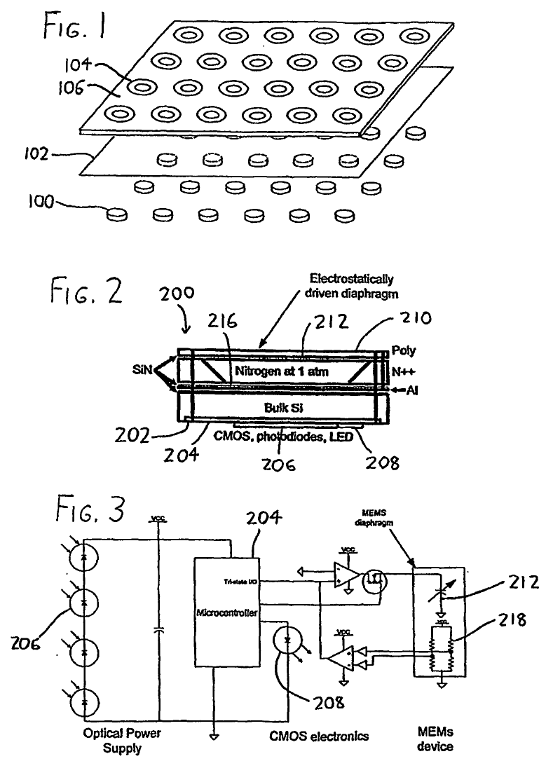

A first exemplary version of the device, as depicted in FIG. 1, involves the implantation of one or more stimulators 100 formed of magnetic material in an array below the skin (with the external surface of the epidermis being depicted by the surface 102), and with the array extending across the area which is to receive the tactile stimulation (e.g., on the abdomen, back, thigh, or other area). Several transmitters 104 are then fixed in an array by connecting web 106 made of fabric or some other flexible material capable of closely fitting above the skin 102 in contour-fitting fashion (with the web 106 being shown above the surface of the skin 102 in FIG. 1 for sake of clarity). The transmitters 104 are each capable of emitting a signal (e.g., a magnetic field) which, when emitted, causes its adjacent embedded stimulator 100 to move. The transmitters 104 may simply take the form of small coils, or may take more complex forms, e.g., forms resembling read/write heads on standard magnetic media data recorders, which are capable of emitting highly focused magnetic beams sufficiently far below the surface 102 to cause the stimulators 100 to move. Thus, when an input signal is applied to a transmitter 104, it is transformed into a signal causing the motion of a corresponding stimulator 100, which is then felt by surrounding nerves and transmitted to the user's brain.

The input signals provided to the transmitters 104 may be generated from camera or microphone data that is subjected to processing (by a computer, ASIC, or other suitable processor) to convert it into desired signals for transmission by the transmitters 104. (Neither the processor, nor the leads to the transmitters 104, are shown in FIG. 1 for sake of clarity). While the signals transmitted by the transmitters 104 could be simply binary on-off signals or gradually varying signals (in which case the user might feel the signals as a step or slow variation in pressure), it is expected that oscillating signals that cause each of the stimulators 100 to oscillate at a desired frequency and amplitude allows a user to learn to interpret more complex information inputs—for example, inputs reflecting the content of visual data, which has shape, distance, color, and other characteristics.

The stimulators 100 may take a variety of forms and sizes. As examples, in one form, they are magnetic spheres or discs, preferably on the order of 2 mm in diameter or less; in another form, they take the form of magnetic particles having a major dimension preferably sized 0.2 mm or less, and which can be implanted in much the same manner as ink particles in tattooing procedures (including injection by air pressure). The stimulators 100 may themselves be magnetized, and may be implanted so their magnetic poles interact with the fields emitted by the transmitters 104 to provide greater variation in motion amplitudes.

It should be understood that each transmitter 104 might communicate signals to more than one stimulator 100, for example, a very dense array of stimulators 100 might be used with a coarse array of transmitters 104, and with each transmitter 104 in effect communicating with a subarray of several stimulators 100. Arrays of stimulators 100 which are denser than transmitter arrays 104 are also useful for avoiding the need for very precise alignment between stimulators 100 and transmitters 104 (with such alignment being beneficial in arrays where there is one transmitter 104 per stimulator 100), since the web 106 may simply be laid generally over the implanted area and each transmitter 104 may simply send its signal to the closest stimulator(s) 100. If precise alignment is needed, one or more measures may be used to achieve such alignment. For example, a particular tactile signal pattern may be fed to the transmitters 104 as the user fits the web 106 over the stimulators 100, with the user then adjusting the web 106 until it provides a sensation indicating proper alignment; and/or certain stimulators 100 may be colored in certain ways, or the user's skin might be tattooed, to indicate where the boundaries of the web 106 should rest. (Recall that if the stimulators 100 are implanted in the dermis, they will be visible through the translucent epidermis in much the same manner as a tattoo unless they are colored in an appropriate fleshtone).

The foregoing version of the invention is “passive” in that the stimulators 100, that are effectively inert structures, are actuated to move by the transmitters 102. However, other versions of the invention wherein the stimulators include more “active” features are may be used, e.g., the stimulators may include features such as mechanical transducers that provide a motion output upon receipt of the appropriate input signal; feedback to the transmitters; onboard processors; and power sources. As in the tactile input system discussed above, these tactile input systems preferably also use wireless communications between implanted stimulators and externally-mounted transmitters. To illustrate, FIGS. 2 and 3 present a second exemplary version of the invention. Here, a stimulator 200 has an external face 202 which includes a processor 204 (e.g., a CMOS for providing logic and control functions), a photocell 206 (e.g., one or more photodiodes) for receiving a wireless (light) signal from a transmitter, and an optional LED 208 or other output device capable of providing an output signal to the transmitter(s) (not shown) in case such feedback is desired. Light send by the transmitter(s) to the photocell 206 both powers the processor 204 and conveys a light-encoded control signal for actuation of the stimulator 200. On the internal face 210 of the stimulator 200, a diaphragm 212 is situated between the dermis or subcutaneous layer and an enclosed gas chamber 214, and an actuating electrode 216 is situated across the gas chamber 214 from the diaphragm 212. Light signals transmitted by the transmitter(s), discussed in greater detail below, are received by the photocell 206, which charges a capacitor included with the processor 204, with this charge then being used to electrostatically deflect the diaphragm 212 toward or away from the actuating electrode 216 when activated by the processor 204. Since the diaphragm 212 only needs to attain peak-to-peak motion amplitude of as little as one micrometer, very little power is consumed in its motion. Piezoelectric resistors (218) (FIG. 3) situated in a Wheatstone bridge configuration on the diaphragm 212 measure the deformation of the diaphragm 212, thereby allowing feedback on its degree of displacement, and such feedback can be transmitted back to the transmitter via output device 208 if desired.

The stimulator 200 is preferably scaled such that it has a major dimension of less than 0.5 mm. With appropriate size and configuration, stimulators 200 may be implanted in the manner of a convention tattoo, with a needle (or array of spaced needles) delivering and depositing each stimulator 200 within the dermis or subcutaneous layer at the desired depth and location. Using state of the MEMS processing procedures, it is contemplated that the stimulator 200 might be constructed with a size as small as a 200 square micrometer face area (e.g., the area across the external face 202 and its internal face 210), with a depth of approximately 70 micrometers. An exemplary MEMS manufacturing process flow for the stimulator 200 is as follows:

| | Side of | |
|---|---|---|
| Step | wafer | Comment |
| 2 um CMOS process | Top | More tolerant to defects |
| Attach handling | Top | |
| wafer | | |
| Planarize (CMP) | Bottom | Thin to approximately 50 um |
| Deposit SiN | Bottom | Insulate lower electrode |
| Sputter Al | Bottom | Lower electrode |
| Lithography | Bottom | Electrode and pads for vias |
| Deposit SiN | Bottom | Insulate lower electrode |
| Deposit poly | Bottom | Approximately 150 um |
| Deposit SiN | Bottom | Mask for cavity |
| Lithography | Bottom | Pattern hole for cavity |
| Etch | — | KOH to form cavity (timed) |
| Deposit poly | Bottom | Seal cavity and strengthen diaphragm |
| Etch (RIE) | Bottom | Vias; 2 through-hole, 1 stops a lower |
| | | electrode metal |
| Fill vias | Bottom | Tungsten |
| Planarize (CMP) | Bottom | Planarize |
| Deposit Ti | Bottom | Titanium (bio-compatible) |
| Lithography | Bottom | Cover only tungsten, or do not do litho |
| | | at all if diaphragm is unaffected |
| Planarize (CMP) | Top | Remove handling wafer |
| Lithography | Top | Pattern for via to pad interconnect |
| Deposit Al | Top | Deposit via a pad interconnect |
| Lithography | Bottom | Pattern for via to pad and via to via |
| | | interconnect |
| Deposit Al | Bottom | Deposit via to pad and via to via |
| | | interconnet |

The transmitter (not shown) may take the form of a flexible electro fluorescent display (in which case it may effectively provide only a single transmitter for all stimulators 200), or it could be formed of an array of LEDs, electro fluorescent displays, or other light sources arrayed across a (preferably flexible) web, as in the transmitter array of FIG. 1. The transmitter(s) supply light to power the photocells 206 of the stimulators 200, with the light bearing encoded information (e.g., frequency and/or amplitude modulated information) which deflects the diaphragms 212 of the stimulators 200 in the desired manner. The light source(s) of the transmitter, as well as the photocells 206 of the stimulator 200, preferably operate in the visible range since photons in the visible range pass through the epidermis for efficient communication with the powering of the stimulators 200 with lower external energy demands.

With appropriate signal tailoring, it is possible to have one transmitter provide distinct communications directed to each of several separate stimulators 200. For example, if the transmitter delivers a frequency modulated signal that is received by all stimulators 200, but each stimulator only responds to a particular frequency or frequency range, each stimulator 200 may provides its own individual response to signals delivered by a single transmitter. An additional benefit of this scheme is that the aforementioned issue of precise alignment between individual transmitters and corresponding stimulators is reduced, since a single transmitter overlaying all stimulators 200 may effectively communicate with all stimulators 200 without being specifically aligned with any one of them.

The description set out above is merely of exemplary versions of the invention. It is contemplated that numerous additions and modifications can be made. As a first example, in active versions of the invention wherein an actuator is used to deliver motion output to the user, actuators other than (or in addition to) a diaphragm 212 may be used, e.g., a piezoelectric bimorph bending motor, an element formed of an electroactive polymer that changes shape when charged, or some other actuator providing the desired degree of output displacement.

As a second example, while the foregoing tactile input systems are particularly suitable for use with their stimulators imbedded below the epidermis, the stimulators could be implemented externally as well, provided the output motion of the stimulators has sufficient amplitude that it can be felt by a user. To illustrate, the stimulators might be provided on a skullcap, and might communicate with one or more transmitters provided on the interior of a helmet.

As an additional example, the foregoing versions of the invention find use with other forms of stimulation, e.g., electrical, thermal, etc., instead of (or in additional to) mechanical stimulation. Greater information is provided in some embodiments by combining multiple types of stimulation. For example, if pressure and temperature sensors are provided in a prosthetic and their output is delivered to a user via mechanical and thermal stimulators, the prosthetic may more accurately mimic the full range of feeling in the missing appendage. As another example, in a vision substitution system, mechanical inputs might deliver information related to the proximity of object (in essence delivering the “contour” of the surrounding environment), and electrical stimulation delivers information regarding color or other characteristics.

The number, size, density, and position (e.g., location and geometry) of stimulators are selected so as to be able to transmit the desired information to the subject for any particular application. For example, where the device is used as a simple alarm, a single stimulator may be sufficient. In embodiments where visual information is provided, more stimulators may be desired. In embodiments where only direction needs to be perceived, a limited ring of stimulators indicating 180-degree, 360-degree direction may be used (or 4 stimulators for N, W, E, S direction, used in combination to indicate intersections). Increase in complexity of information with a limited set of stimulators may also be achieved by varying gradients of signal (intensity, pitch, spatial attribute, depth) to create a palette of tactile “colors” or sensations (e.g., paraplegics perceive one level of gradient as a “bladder full” alarm and another level of gradient with the same stimulator or stimulators as a “object in contact with skin” perception).

The tactile input systems of the present invention can be used for sensory substitution and/or enhancement, e.g., to provide tactile input to supplement or replace visual and/or audible input or other natural or non-natural sensory information. Systems and methods for receiving external information (e.g., audio, video, etc.) and translating said information into patterns that can be imparted to a subject by an array of stimulators are described, for example, in U.S. Pat. No. 6,430,450. Various other methods of encoding externally collected data to obtain tactile stimulation schemes appear in the literature relating to surface tactile displays and may be used with the systems and methods of the present invention. As an example of the use of the systems for the substitution or augmentation of the user's visual system, a two dimensional array of stimulators may be implanted at a desired location on the user's body, and an external web or “pack” of one or more transmitters may be placed against the skin at this location. A camera or similar imaging device is then used to capture visual information (either continuously or discretely, e.g., at times selected by the user), with a signal processor on the web or pack (or elsewhere) converting the visual signal into tactile signals for transmission to the stimulators. The tactile input system thereby substitutes for or augments an impaired visual system (e.g., in the case where the user has partial or complete blindness), or substitutes for or augments unimpaired visual systems as well. For example, in some embodiments, the system provides “rear vision” and/or enhanced peripheral vision to operators of motor vehicles (e.g., cars, planes, boats, spacecraft, etc.); provides “night vision” (e.g., from an infrared input) to supplement the user's standard vision in low light conditions; or provides images taken in other non-visible wavelengths to effectively allow a user to “see” outside of the normal spectra.

The tactile input systems can similarly be used to substitute for or augment existing audio systems. As an example, in some embodiments, the surface of a user's ear canal is implanted with numerous stimulators, and an appropriate number of transmitters are provided in a removable Completely in the Canal (CAC) plug, much like a compact hearing aid, which is placed in the ear canal to provide power and communications to the stimulators. The plug may house, for example, a microphone and processor to sense sound and sort/transform the sound into individual frequency binned signals that represent the pitch or frequency content of the sound captured by the microphone. Selected stimulators are then actuated in accordance with the frequency components of the captured sound, in a manner analogous to the process whereby the ear's cochlea provides signals to the brain. With training the user automatically associates particular tactile patterns from the implanted array with sound information, such that the brain automatically perceived the information and, for example, integrates the information into its general sensory perception (e.g., integrates the information with naturally perceived sound to provide more or better “hearing”). As with the exemplary visual-to-tactile input systems discussed above, the audio-to-tactile input systems, in some embodiments, supply tactile input for audio inputs outside of the ordinary audible range, or provide audio-like inputs in response to non-audio signals (e.g., provide an audio-like tactile input in response to visible or other inputs).

Senses other than vision and hearing can also be replaced or augmented as well. For example, a simulated sense of taste can be generated by implementing the tactile input system in conjunction with a device for sensing chemical concentrations in the air, allowing a user to “feel” the concentration of pollutants or hazardous fumes. Alternatively or additionally, the invention can simply be used to compensate for existing tactile impairment, e.g., insensate feet (as might result from complications of diabetes) can be equipped with one or more of pressure and temperature sensors, with the output of these sensors being sent (with or without processing) to one or more transmitters situated adjacent a stimulator array elsewhere on the body (e.g., a different body part). As a result, the user's sense of touch on his/her foot or feet is effectively moved elsewhere on his/her body. This ability to “transport” tactile input to other areas allows, for example, for a prosthetic limb to simulate sensation: sensors in the prosthetic can communication their signals to one or more transmitters (e.g., located in the socket/fitting of the prosthetic), which in turn communicate with stimulators implanted in the body (e.g., near the stump fit into the socket/fitting).

In some embodiments, the implanted components further serve aesthetic and/or entertainment purposes. Because the embedded components are, or can be designed to be, visible, they may be used to serve tattooing or cosmetic implant functions—i.e., to provide color, texture, and/or shapes under the skin with desired aesthetic features. Additional embedded components without sensory function may be added to enhance or fill out the image provided by the embedded stimulators. LED or other components can provide light to enhance the appearance of the device. For example, stimulators that are in use may be lit. Alternatively lighting patterns are provided randomly or upon cue (e.g., as a timekeeping device, upon receipt of a signal from an external device (e.g., phone)).

In some embodiments, a large number of stimulators are provided all over the body to effectively provide a tactile body “suit” that permits a diverse range of tactile stimulation at one or more body parts, receiving any of a wide variety of information from external sources. Corresponding transmitters of one or more types may be fitted into clothing or other coverings to provide the ability to activate any one or more the stimulators as desired.

The implanted stimulators of the present invention may also be used in conjunction with external stimulators to provide a more advanced tactile input system.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields, are intended to be within the scope of the following claims.

We claim:

1. A tactile input system comprising multiple stimulators implanted in the skin of a subject below the epidermis in a closely spaced array, wherein the stimulators or a portion thereof are independently configured to deliver a tactile stimulation.

2. The system of claim 1, wherein said tactile stimulation is mechanical stimulation.

3. The system of claim 1, wherein said tactile stimulation is electrical stimulation.

4. The system of claim 1, wherein said tactile stimulation is thermal stimulation.

5. The system of claim 1, wherein said stimulators are configured to provide said tactile stimulation in response to a wireless signal.

6. The system of claim 5, wherein said wireless signal comprises a light signal.

7. The system of claim 1, wherein said stimulators are provided with a biocompatible coating.

8. The system of claim 1, further comprising a transmitter configured to transmit a signal to one or more of said stimulators to initiate said tactile stimulation.

9. The system of claim 1, wherein said stimulators individually have a volume of less than 10 cubic millimeters.

10. The system of claim 1, wherein said stimulators comprise a movable diaphragm.

11. The system of claim 1, wherein said stimulators are not in direct or indirect physical contact with each other.

12. An implantable tactile input system comprising multiple stimulators configured to be implanted in the skin of a subject below the epidermis in a closely spaced array, wherein the stimulators or a portion thereof are independently configured to deliver a tactile stimulation to said subject when implanted.

13. The system of claim 12, wherein said tactile stimulation is mechanical stimulation.

14. The system of claim 12, wherein said tactile stimulation is electrical stimulation.

15. The system of claim 12, wherein said tactile stimulation is thermal stimulation.

16. The system of claim 12, wherein said stimulators are configured to provide said tactile stimulation in response to a wireless signal.

17. The system of claim 16, wherein said wireless signal comprises a light signal.

18. The system of claim 12, wherein said stimulators are provided with a biocompatible coating.

19. The system of claim 12, further comprising a transmitter configured to transmit a signal to one or more of said stimulators to initiate said tactile stimulation.

20. The system of claim 12, wherein said stimulators individually have a volume of less than 10 cubic millimeters.

21. The system of claim 12, wherein said stimulators comprise a movable diaphragm.

22. The system of claim 12, wherein said stimulators are not in direct or indirect physical contact with each other.

23. A method for imparting information to a subject comprising: transmitting a signal from a transmitter to multiple stimulators implanted in the skin of said subject under conditions such that said stimulators provide a tactile stimulation that conveys information from said signal to the brain of said subject.

24. The method of claim 23, wherein said stimulators are implanted below the epidermis in a Closely spaced array.

25. The method of claim 23, wherein said stimulators or a portion thereof are independently configured to deliver said tactile stimulation.

26. The method of claim 23, wherein said tactile stimulation is mechanical stimulation.

27. The method of claim 23, wherein said tactile stimulation is electrical stimulation.

28. The method of claim 23, wherein said tactile stimulation is thermal stimulation.

29. The method of claim 23, wherein said stimulators are configured to provide said tactile stimulation in response to a wireless signal.

30. The method of claim 29, wherein said wireless signal comprises a light signal.

31. The method of claim 23, wherein said information comprises visual information.

32. The method of claim 23, wherein said information comprises audio information.

33. The method of claim 23, wherein said information comprises environmental information.

34. The method of claim 23, wherein said information comprises tactile information from a body location other than the location where said multiple stimulators are implanted.
